(19) United States
(12) Patent Application Publication
(10) Pub. No.: US 2010/0254933 A1
(43) Pub. Date: Oct. 7, 2010

(54) COSMETIC COMPOSITION CONTAINING A POLYAMIDE RESIN, A GLYCERYL ESTER AND AN APOLAR WAX

(75) Inventors: Sophie Favre, Paris (FR); Monique Narebski, La Garenne Colombes (FR); Marion Meunier, Lyon (FR)

Correspondence Address: YOUNG & THOMPSON, 209 Madison Street, Suite 500, Alexandria, VA, 22314, US

(73) Assignee: CHANEL PARFUMS BEAUTE, Neuilly sur Seine Cedex (FR)

(21) Appl. No.: 12/746,620

(22) PCT Filed: Dec. 5, 2008
(86) PCT No.: PCT/EP08/66911

§ 371 Date: Jun. 7, 2010

(30) Foreign Application Priority Data
Dec. 6, 2007 (FR) 07 08517

(51) Int. Cl. A61K 8/92; A61Q 1/00

(52) U.S. Cl. 424/78.03

(57) ABSTRACT

A cosmetic composition includes, in a physiologically acceptable medium: (a) at least one polyamide resin bearing end groups with a tertiary amide or ester function; (b) at least one ester obtained from: (i) a saturated or unsaturated, linear or branched, aliphatic acid or hydroxy acid containing from 8 to 30 carbon atoms, (ii) a linear or branched diacid containing from 12 to 36 carbon atoms, and (iii) glycerol or a glycerol condensate; and (c) at least one apolar wax. It also relates to a cosmetic process for making up the lips, including the topical application of this composition to the lips.

The present invention relates to a cosmetic composition. It also relates to a cosmetic process for making up the lips or the eyelids, comprising the topical application of this composition to the lips or the eyelids.

Among lip makeup compositions, “glosses” denote compositions that are generally fluid and transparent to opalescent, intended to give the lips a gloss through the use of certain oils having a high refractive index. These oils are structured or gelled with waxes or oily gelling agents which make it possible to adjust the viscosity of the composition. The compositions thus obtained are generally packaged in a container such as a small bottle provided with an applicator, generally a foam brush.

It has been observed that lip glosses, just like certain lipsticks, have a tendency to migrate, after application to the lips, to the area around the mouth and in particular into the perilabial wrinkles. This phenomenon is particularly visible in the case of products intended to give the lips not only a gloss but also a strong color, and which consequently contain a high amount of dyestuffs. Now, this migration phenomenon is obviously undesirable since one of the first qualities that lip makeup products must satisfy is that of obtaining a sharp colored mouth outline.

It is, admittedly, known that recourse to structuring agents of wax and/or filler type makes it possible to substantially reduce these migration problems. However, these structuring agents negatively affect the slip of the composition on the lips and have a tendency to produce a matt effect which is not desirable for lip glosses.

Another solution envisioned up until now for reducing this migration phenomenon has consisted in using certain oily gelling agents of polyamide structure. The products of the Sylvaclear® range provided by the company Arizona are thus proposed for the formulation of cosmetic gloss products in which they not only offer good pigment dispersion but for which they also contribute to improving the water resistance and transfer resistance and to reducing migration. However, it has been observed that the use of these oily gelling agents does not make it possible sufficiently to reduce the migration of lip makeup products to the area around the mouth, which remains of the order of a few millimeters. Since a reduction in this migration effect of one millimeter is already very perceptible to the consumer, it would therefore be desirable to be able to have lip makeup products that migrate less than the known products, or do not even migrate at all.

Similarly, it would be useful to have eye makeup products, such as eyeliners or eye shadows, which do not migrate substantially into the folds of the eyelids or into the fine lines around the eye.

To the applicant's credit, it has, after numerous experiments, succeeded in developing a combination of fatty-phase structuring agents making it possible to reduce or even prevent the migration of makeup products, without substantially affecting the gloss of the makeup obtained. The applicant has also demonstrated that the compositions according to the invention make it possible to increase the fastness of the makeup, and also to reduce the phenomenon of threading coming out of the small bottle, which is sometimes observed with lip glosses, and which is reflected by the formation of a thread, consisting of the makeup composition between the tip of the brush and the opening of the small bottle. All these properties contribute to guaranteeing a sharp and precise application of the makeup composition, in particular to the lips, and to maintaining a sharp outline of the colored lips throughout the day.

A subject of the present invention is therefore a cosmetic composition comprising, in a physiologically acceptable medium:
(a) at least one polyamide resin bearing end groups with a tertiary amide or ester function;
(b) at least one ester obtained from: (i) a saturated or unsaturated, linear or branched, aliphatic acid or hydroxy acid containing from 8 to 30 carbon atoms, (ii) a linear or branched diacid containing from 12 to 36 carbon atoms, and (iii) glycerol or a glycerol condensate; and
(c) at least one apolar wax.

### Polyamide Resin

The first constituent of the composition according to the invention is a polyamide resin bearing end groups with an ester and/or amide function.

According to one preferred embodiment of the invention, the polyamide resin comprises one or more copolymers corresponding to formula (1):

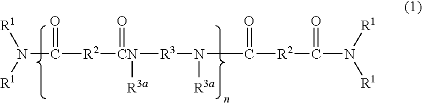

in which:

n denotes a number of repeating units such that the end amide groups represent from 10% to 50% of the total number of amide groups of the resin,

the R¹ groups independently denote C₁-C₂₂ hydrocarbon-based, in particular alkyl or alkenyl, groups,

the R² groups independently denote C₂-C₄₂ hydrocarbon-based, in particular alkylene or alkenylene, groups,

the R³ groups independently denote organic groups containing at least two carbon atoms and optionally one or more oxygen and/or nitrogen atoms, and

the R^{3a} groups independently denote hydrogen, C₁-C₁₀ alkyl groups or a direct bond with R³, or together form a heterocycle including the nitrogen atoms to which they are attached and R³.

According to another embodiment of the invention, the polyamide resin comprises one or more copolymers corresponding to formula (2):

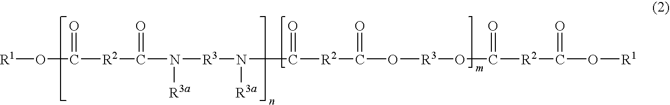

in which:

n denotes a number of repeating units such that the end ester groups represent from 10% to 50% of the total number of amide and ester groups of the resin,

the R¹ groups independently denote C₁-C₂₂ hydrocarbon-based, in particular alkyl or alkenyl, groups,

the R² groups independently denote C₂-C₄₂ hydrocarbon-based, in particular alkylene or alkenylene, groups,

the R³ groups independently denote organic groups containing at least two carbon atoms and optionally one or more oxygen and/or nitrogen atoms, and

the R^{3a} groups independently denote hydrogen, C₁-C₁₀ alkyl groups or a direct bond with R³, or together form a heterocycle including the nitrogen atoms to which they are attached and R³.

In formulae (1) and (2) above, n preferably ranges from 1 to 10 and more preferably from 1 to 5.

In addition, the end amide or ester groups preferably represent from 15% to 40%, preferably from 20% to 35% of the total number of amide and ester groups of the resin.

According to one preferred embodiment of the invention:
at least one, and preferably all, of the R¹ groups is (are) alkyl or alkenyl groups containing from 4 to 22 carbon atoms and preferably from 16 to 22 carbon atoms, and/or at least one, and preferably at least 50% or even all, of the R² groups is (are) alkylene or alkenylene groups containing from 2 to 42, and preferably from 30 to 42 carbon atoms.

Moreover, according to the invention, R^{3a} preferably denotes hydrogen. In this case, at least one, and preferably all, of the R³ groups may contain from 2 to carbon atoms, preferably from 2 to 12, and more preferably from 2 to 8 carbon atoms. R³ may thus denote a polyoxyalkylene group such as a polyoxyethylene or polyoxypropylene group. As a variant, R³ may denote a polyalkylene amine group such as an —NH—(CH₂CH₂NH)ₚCH₂CH₂—NH— group where p is an integer from 1 to 5.

According to another embodiment, R^{3a} may denote a C₁₋₃ alkyl group, such as a methyl, ethyl, n-propyl or isopropyl group.

According to yet another embodiment, the R^{3a} groups may form, together and with R³ and the adjacent nitrogen atoms, a piperazine ring.

The resin used according to the invention may, for example, have a weight-average molecular mass ranging from 500 to 10 000 g/mol, preferably from 2000 to 6000 g/mol, more preferably from 4000 to 6000 g/mol, and better still from 4000 to 5000 g/mol, as measured by gel permeation chromatography using calibration with polystyrene. In addition, it preferably has a number-average molecular mass ranging from 2000 to 4000 g/mol and more preferably from 2500 to 3500 g/mol.

It may in particular be prepared as described in document US 2003/0069388, the content of which is incorporated herein by way of reference.

Examples of polyamide resins with tertiary amide endings are those identified by the INCI name “Ethylenediamine/Hydrogenated Dimer Dilinoleate Copolymer Bis-Di-C14-18 Alkyl Amide” and available, for example, under the trade name Sylvaclear® A200V or Sylvaclear® A2614V from the company Arizona Chemical. An example of a polyamide resin with ester endings is that identified by the INCI name “Bis-stearyl Ethylenediamine/Neopentyl Glycol/Stearyl Hydrogenated Dimer Dilinoleate Copolymer” and available, for example, under the trade name Sylvaclear® C75V from the company Arizona Chemical.

The polyamide resin may represent, for example, from 0.01% to 30% by weight, preferably from 1% to 20% by weight, more preferably from 5% to 15% by weight, and better still from 8% to 12% by weight, relative to the total weight of the composition.

### Glyceryl Ester

The composition according to the invention contains, as second essential constituent, at least one ester obtained from: (i) a saturated or unsaturated, linear or branched, aliphatic acid or hydroxy acid containing from 8 to 30 and preferably from 12 to 22 carbon atoms, (ii) a linear or branched diacid containing from 12 to 36 and preferably from 12 to 20 carbon atoms, and (iii) glycerol or a glycerol condensate.

The aliphatic acid (i) in particular may be chosen from lauric acid, myristic acid, palmitic acid, oleic acid, stearic acid, 12-hydroxystearic acid and behenic acid, and mixtures thereof. In addition, the diacid (ii) may, for example, be chosen from eicosadioic acid, ethyloctadecanedioic acid and dodecanedioic acid, and mixtures thereof.

A glyceryl ester of this type, preferred for use in the present invention, is the diester of eicosadioic acid and of glycerol esterified with behenic acid. This compound is an ester of formula RO—CH₂—CH(OR)—CH₂—O—OC—(CH₂)₁₈—CO—O—CH₂—CH(OR)—CH₂—OR, where the R groups independently denote a —CO—(CH₂)₂₀—CH₃ group or H, on the condition that at least one of the R groups is other than H, or a mixture of such esters. It is in particular available from the company Nisshin Oillio under the trade name Nomcort® HK-G.

The ester (b) may represent from 0.01% to 30% by weight, preferably from 1% to 20% by weight, more preferably from 5% to 15% by weight, and better still from 8% to 12% by weight, relative to the total weight of the composition.

### Apolar Wax

The third essential constituent of the composition according to the invention is an apolar wax.

The term “wax” is intended to mean a fatty substance with a reversible liquid/solid change in state, which has a melting point above 30° C. and generally below 90° C. and which is liquid under the conditions for preparing the composition and has an anisotropic crystalline organization in the solid state. The term “apolar” is intended to mean a wax containing only carbon, hydrogen and/or phosphorus atoms and in particular a hydrocarbon.

Examples of apolar waxes are in particular paraffin wax, polymethylene wax, polyethylene wax, polypropylene wax or ethylene/propylene copolymer wax, micro-crystalline waxes and ozokerite, and mixtures thereof. Examples of such waxes are in particular sold by the company Sasol Wax under the trade name Sasol Wax® C80, by the company Jeen under the trade name Jeenate® 2H to 6H and by the company New Phase under the trade names Performalene® 500 and Performa® V343. In general, it is preferred to use a wax which has a melting point below 80° C., preferably below 70° C., for example between 50 and 70° C. Polyethylene waxes are preferred for use in the present invention.

The apolar wax may represent from 0.01% to 40%, preferably from 1% to 30%, more preferably from 5% to 20%, and better still from 8% to 12% by weight, relative to the total weight of the composition.

### Other Ingredients

Apart from the abovementioned three constituents, the composition according to the invention may contain various ingredients, such as oils, polar waxes, lipophilic thinning agents, pasty compounds and/or film-forming polymers.

The oils may be chosen from silicone oils, linear or branched hydrocarbons of mineral or synthetic origin, synthetic (poly)esters and (poly)ethers and in particular (poly) esters of C₆-C₂₀ acids and of C₆-C₂₀ alcohols which are advantageously branched, such as isononyl isononanoate, plant oils, branched and/or unsaturated fatty acids, branched and/or unsaturated fatty alcohols, and fluorooils and also mixtures thereof.

In the present invention, at least one glossy oil, i.e. an oil having a refractive index of greater than 1.45, and preferably greater than 1.47, is preferably used.

Examples of glossy oils are especially natural oils and in particular castor seed oil; mono- and polyesters of fatty acids and/or of fatty alcohols, the fatty chain of which contains from 6 to 20 carbon atoms, and in particular: mono- and polyesters of hydroxy acids and of fatty alcohols, such as diisostearyl malate, esters of benzoic acid and of fatty alcohols, such as C₁₂-C₁₅ alkyl benzoate, polyesters of polyols and in particular (di)pentaerythrityl polyesters, such as pentaerythrityl tetraisostearate, dipentaerythrityl pentaisononanoate and dipentaerythrityl C₅-C₉ esters, or of polyglycerol, such as polyglyceryl-2-triisostearate or that known under the INCI name “bis-diglyceryl polyacyladipate-1” and sold by the company Sasol under the trade name Softisan® 645, or of trimethylolpropane, such as trimethylolpropane triethylhexanoate, which is in particular sold by the company Kokyu Alcohol Kogyo under the trade name KAK TTO®, or of propylene glycol, such as propylene glycol dibenzoate, which is in particular sold by the company Inolex under the trade name Lexfeel Shine®, and isocetyl stearoyl stearate; and polyesters of hydrogenated castor oil, such as the esters sold by the company Kokyu Alcohol Kogyo under the trade names Risocast® DA-H and Risocast® DA-L.

Although this variant is less preferred, it is possible to use, as a variant or in addition to the abovementioned glossy oils, fluorinated or phenylated silicones.

In addition, it is clearly understood that the composition according to the invention may comprise mixtures of the oils mentioned above.

The composition according to the invention may also contain at least one polar wax chosen in particular from animal waxes, plant waxes and synthetic waxes or silicone waxes containing polar groups such as esters. Mention may thus be made of carnauba wax, candelilla wax, beeswax (Cera alba), Chinese insect (Ericerus pela) wax; Japan wax, sumac wax, montan wax, triesters of C₈-C₂₀ acids and of glycerol, such as glyceryl tribehenate, the acetylated glycol stearate sold in particular by the company Vevy under the trade name Cetacene®, and mixtures thereof. These waxes may in particular be used in a form predispersed in an oil, as is the case of the mixture of candelilla wax and jojoba seed oil sold by the company Ina Trading under the trade name Green Grease®.

As a variant or in addition, the composition according to the invention may contain at least one lipophilic gelling agent other than the polyamide resin described above. Examples of lipophilic gelling agents are especially silicone polymers and more particularly organopolysiloxane elastomers. Among these, mention may be made of at least partially crosslinked polymers resulting from the reaction of an organopolysiloxane bearing unsaturated groups, such as vinyl or allyl groups, located at the end or in the middle of the chain, preferably on a silicon atom, with another reactive silicone-based compound such as an organohydrogenopolysiloxane. These polymers are usually available in the form of a gel in a volatile or nonvolatile silicone-based solvent or in a hydrocarbon-based solvent. Examples of such elastomers are especially sold by the company Shin Etsu under the trade names KSG-6, KSG-16, KSG-31, KSG-32, KSG-41, KSG-42, KSG-43 and KSG-44, and by the company Dow Corning under the trade names DC 9040 and DC 9041. Another oily gelling agent is composed of a silicone polymer obtained by self-polymerization of an organo-polysiloxane functionalized with epoxy and hydrosilylated groups in the presence of a catalyst, which is commercially available from the company General Electric under the trade name Velvesil® 125. Another lipophilic gelling agent is composed of a noncrosslinked cyclic vinyldimethicone/dimethicone copolymer such as that sold by the company Jeen under the trade name Jeesilc® PS (including PS-VH, PS-VHLV, PS-CM, PS-CMLV and PS-DM). The lipophilic gelling agent may, as a variant, be a bentone or a sucrose ester such as that denoted by the INCI name “Sucrose tetrastearate triacetate”.

The composition according to the invention may also contain one or more pasty compounds, i.e. lipophilic fatty substances which, like the waxes, are capable of undergoing a reversible liquid/solid change in state and have, in the solid state, an anisotropic crystalline organization, but which differ from the waxes in that they contain, at a temperature of 23° C., a liquid fraction and a solid fraction.

It may also comprise at least one film-forming polymer capable of introducing hold and/or transfer-free properties and/or gloss to the makeup conferred by the composition. It may in particular be an optionally urethane- or fluoro- or acrylate-modified silicone-based polymer such as the (meth)acrylate silicones sold by Shin Etsu under the trade names KP-545, KP-561 and KP-562, or the polymers sold by the company Dow Corning under the trade names DC FA® 4002 ID and DC FA® 4001 CM. Other examples of film-forming polymers are silicone resins and in particular MQ resins, such as trimethylsiloxysilicates, and MT resins such as silsesquioxane derivatives and in particular polymethylsilsesquioxanes, sold in particular by the company Shin Etsu, and also the polypropylsilsesquioxane sold by the company Dow Corning under the trade name DC® 670 or the phenylpropylpolysilsesquioxane sold by the company Wacker under the trade name Belsil® SPR45VP. Another example is composed of fluorosilicone-based polymers identified by the INCI name trifluoropropyldimethylsiloxy triethylsiloxysilicate, such as that sold by the company General Electric under the trade name XS66-B8226®. Use may also be made, as film-forming polymers, of bioadhesive polymers obtained, for example, by polycondensation of dimethiconol and of MQ silicate resin in a solvent such as heptane, which are sold in particular by the company Dow Corning under the trade names DC7-4405® low tack and DC7-4505® high tack. Other examples of film-forming polymers are cyclic or noncyclic polyolefins such as polybutene (Indopol® H-1900 and H-2100, for example) or polycyclopentadiene, in particular sold by the company Kobo under the trade name Koboguard® 5400, or else polydicyclopentadiene. Yet other examples of film-forming polymers are composed of copolymers of vinylpyrrolidone (VP) and/or of linear olefins, such as VP/hexadecene and VP/eicosene copolymers, including Antaron® V216 and V220 from the company ISP or else ethylene/vinyl acetate copolymers, such as AC 400® from the company Baerlocher. Mention may also be made of polyethers such as the poly(vinyl stearyl ether) sold in particular by the company Phoenix under the trade name Giovarez® 1800. Other film-forming polymers that can be used in this invention are polyacrylates, such as the poly(ethyl acrylate) copolymers sold in particular by the company Creations Couleurs under the trade name Creasil® 7 ID.

It is preferable for the composition according to the invention to be anhydrous, i.e. for it to contain no water, or at the very most the water naturally included in the ingredients that it contains, i.e. at most 3% by weight of water, or even at most 1% by weight of water. However, as a variant, this composition may be in the form of a water-in-oil or oil-in-water emulsion containing, in addition to the fatty phase described above, an aqueous phase containing water and optionally, water-miscible solvents such as alcohols or glycols and/or hydrophilic additives such as hydro-philic gelling agents and/or hydrophilic active agents.

In the case where it is in the form of an emulsion, it is clearly understood that the composition according to the invention may also contain one or more water-in-oil emulsifiers preferably chosen from nonionic surfactants, such as the polyethoxylated (30 EO) dipolyhydroxystearate sold in particular under the trade name Arlacel® P135 by the company Uniqema or polyglyceryl polyesters such as polyglyceryl-4 diisostearate/polyhydroxystearate/sebacate (Isolan GPS® from Goldschmidt); or else one or more oil-in-water emulsifiers, chosen from nonionic surfactants, such as optionally polyethoxylated sorbitan esters, fatty acid esters of glycerol, fatty acid esters of sucrose, fatty acid esters of polyethylene glycol, polyether-modified polysiloxanes, fatty alcohol ethers of polyethylene glycol, alkyl polyglycosides, hydrogenated lecithin and hydrophobically modified saccharides of fructan type, such as inulin lauryl carbamate (Inutec SP1® from Orafti).

The composition used according to the invention may also contain at least one filler. This term is intended to mean any inorganic or organic particle of any shape (in particular spherical or lamellar) which is insoluble in the composition. Examples of fillers are talc, mica, silica, kaolin, boron nitride, starch, starch modified with octenylsuccinic anhydride, polyamides, silicone resins, silicone elastomer powders and acrylic polymer powders, in particular poly(methyl methacrylate) powders. The fillers may, in particular, be composed of several layers of different chemical nature and/or of different physical form, and may in particular be in the form of lamellae coated with spherical fillers. They may be modified using various surface treatments. An example of a surface-treated filler consists of the silica modified with an ethylene/methacrylate copolymer, sold in particular by the company Kobo under the trade names DSPCS® 20N-I2, 3H-I2 and I2.

The composition may also contain at least one dyestuff chosen from water-soluble or liposoluble dyes, fillers having the effect of coloring and/or opacifying the composition and/or of coloring the lips, such as pigments, pearlescent agents, lakes (water-soluble dyes adsorbed onto an inert inorganic carrier) and mixtures thereof. These dyestuffs may be optionally surface-treated with a hydrophobic agent, such as silanes, silicones, fatty acid soaps, C₉-C₁₅ fluoroalkyl phosphates, acrylate/dimethicone copolymers, mixed C₉-C₁₅ fluoroalkyl phosphate/silicone copolymers, lecithins, carnauba wax, polyethylene, chitosan and optionally acylated amino acids, such as lauroyl lysine, disodium stearoyl glutamate and aluminum acyl glutamate. The pigments may be inorganic or organic, natural or synthetic. Examples of pigments are in particular iron oxides, titanium oxides or zinc oxides, and also composite pigments and goniochromatic, pearlescent, interferential, photochromic or thermochromic pigments, without this list being limiting. Particularly preferred examples of pigments that can be used in the composition according to the invention are hemispherical composite pigments produced from crosslinked poly(methacrylic acid) methyl ester and from organic dyes. Such composite pigments are in particular sold by the company Daito Kasei under the trade name 3D Tech®. The pearlescent agents may be chosen from those conventionally present in makeup products such as mica/titanium dioxide products. As a variant, they may be pearlescent agents based on mica/silica/titanium dioxide, or based on synthetic fluorophlogopite/titanium dioxide (Sunshine® from Maprecos), on calcium sodium borosilicate/titanium dioxide (Reflecks® from Engelhard) or on calcium aluminum borosilicate/silica/titanium dioxide (Ronastar® from Merck).

Advantageously, when it contains one or more pigments, the composition according to the invention also contains at least one dispersant such as butylene glycol cocoate.

The composition according to the invention may also contain one or more sweetening agents, such as sorbitol, sucrose, xylitol, acesulfame K and sodium saccharinate; antioxidants such as alkylated or phosphorylated esters of ascorbic acid, or else tocopherol and its esters, sequestering agents such as EDTA salts; pH modifiers; preservatives and fragrances.

It may also contain at least one UV screen chosen from organic and inorganic screens and mixtures thereof. As organic screens, mention may in particular be made of dibenzoylmethane derivatives (including butyl methoxydibenzoylmethane), cinnamic acid derivatives (including ethylhexyl methoxycinnamate), salicylates, para-aminobenzoic acids, β,β′-diphenylacrylates, benzophenones, benzylidenecamphor derivatives, phenylbenzimidazoles, triazines, phenylbenzotriazoles and anthranilic derivatives. As inorganic screens, mention may in particular be made of screens based on inorganic oxides in the form of pigments or of nanopigments, which may be coated or uncoated, and in particular based on titanium dioxide or on zinc oxide.

Examples of such adjuvants are mentioned in particular in the CTFA dictionary (International Cosmetic Ingredient Dictionary and Handbook published by The Cosmetic, Toiletry and Fragrance Association, 11th edition, 2006).

### Method of Manufacture

The composition according to the invention may be in the form of a solid product, such as a cast product in a dish, or in the form of a stick or in semi-solid form, such as a cream or a syrup packaged, for example, in a small bottle, the stopper of which is provided with an applicator or in a tube, the orifice of which is provided with an applicator such as a foam pad, or else in a pot or a dish. The applicator is usually made of foam, typically in the form of a fine brush or a coarse brush. An applicator of felt type may be used as a variant.

It may be manufactured according to a method comprising the following steps:
mixing of the three structuring agents according to the invention with oils, with stirring, at a temperature of at least 80° C., preferably of at least 90° C., for example in a deflocculating or centrifuging deflocculating turbine (Rayneri or Moritz type) conventionally used for the manufacture of lipsticks,
addition of dyestuffs, optionally pre-milled in an oil,
homogenization of the mixture obtained,
either hot-casting of the mixture so as to form a solid composition after cooling, optionally followed by destructuring of the solid composition thus obtained so as to form a viscous paste, or cooling of the mixture with stirring (for example in the abovementioned turbines) to ambient temperature so as to prevent recrystallization of the waxes and to obtain a semi-solid composition.

The destructuring step makes it possible in particular to obtain cosmetic products of gloss type. It may in particular involve an extrusion step, for example in a twin-screw extruder (in particular of Clextral BC21 type) or in a cooker-extruder mixer, or a milling step in a roller mill (such as an Exakt 50 three-roll mill) or a (micro)bead mill (for example of Bühler K8 type), the use of a roller mill being preferred.

As indicated above, such a semi-solid composition may, as a variant, be obtained by cooling the mixture of components obtained in the first step described above, with stirring.

The method for manufacturing this composition preferably comprises a destructuring step.

### Uses

Preferably, the composition used according to the invention is employed as a lip makeup product, for example, as a lip gloss or as a lipstick. As a variant, it may be used as an eye makeup product and in particular as an eye shadow or eyeliner, or even as a mascara. As another variant, the composition according to the invention may be used as a skin makeup product, such as a foundation.

A subject of the present invention is therefore also a cosmetic process for making up the lips comprising the topical application to the lips of the composition as described above.

The invention will now be illustrated by the following nonlimiting examples which are given only for the purposes of illustration and are not intended to limit the scope of the invention, which is defined by the attached claims.

### EXAMPLES

### Example 1

### Lip Gloss

A lip gloss having the composition indicated in Table 1 below, in which the proportions of the ingredients are expressed as percentages by weight, was manufactured.

TABLE 1

| Phase | INCI NAME/chemical name | Amount (%) |
|---|---|---|
| 1 | ETHYLENEDIAMINE/HYDROGENATED | 10.56 |
| | DIMER DILINOLEATE COPOLYMER | |
| | BIS-DI-C14-18 ALKYL AMIDE | |
| | GLYCERYL BEHENATE/EICOSADIOATE | 10.56 |
| | SYNTHETIC WAX | 10.56 |
| | DIETHYLHEXYL ADIPATE | 3.00 |
| | DIISOSTEARYL MALATE | 13.70 |
| | OCTYLDODECANOL | 23.50 |
| | PROPYL PARABEN | 0.30 |
| | PHENOXYETHANOL | 0.10 |
| 2 | POLYBUTENE | 7.50 |
| 3 | Lakes | 1.26 |
| | | 1.08 |
| | | 2.00 |
| | | 1.68 |
| | DIISOSTEARYL MALATE | 14.20 |

This composition was prepared in the following way:

The ingredients of phase 1 were melted at 95° C. with stirring, before incorporating therein phase 2 and then phase 3 milled beforehand in a roller mill (3 passages). The liquid mixture thus obtained was hot-cast. After cooling, a solid hard paste was obtained, which was destructured at ambient temperature in a roller mill (just 1 passage, position 3). The glossy viscous paste thus obtained was subsequently packaged in small bottles provided with an applicator.

### Example 2

### Eye Shadow

An eye shadow having the composition indicated in Table 2 below, in which the proportions of the ingredients are expressed as percentages by weight, was manufactured.

TABLE 2

| Phase | INCI NAME | (%) |
|---|---|---|
| 1 | ISODODECANE | 45.25 |
| | SILICA SILYLATE | 2 |
| 2 | ETHYLENEDIAMINE/HYDROGENATED | 18.75 |
| | DIMER DILINOLEATE COPOLYMER | |
| | BIS-DI-C14-18 ALKYL AMIDE | |
| | NEOPENTYL GLYCOL DIOCTANOATE | 10 |
| 3 | GLYCERYL BEHENATE/EICOSADIOATE | 3 |
| | SYNTHETIC WAX | 1 |
| 4 | MICA & IRON OXIDES & TITANIUM | 20 |
| | DIOXIDE | |

This composition may be prepared in the following way:

The isododecane was cold-gelled with the silica, with stirring. The ingredients of phases 1 to 3 were subsequently mixed at 90° C. with stirring. Phase 4 was then incorporated therein so as to obtain a homogeneous mixture which was hot-cast into dishes.

### Example 3

### Eyeliner

An eyeliner having the composition indicated in Table 3 below, in which the proportions of the ingredients are expressed as percentages by weight, was manufactured.

TABLE 3

| Phase | INCI NAME | % |
|---|---|---|
| 1 | ETHYLENEDIAMINE/HYDROGENATED | 20.5 |
| | DIMER DILINOLEATE COPOLYMER | |
| | BIS-DI-C14-18 ALKYL AMIDE | |
| | ISODODECANE | 8.55 |
| | BEESWAX | 1 |
| | GLYCERYL BEHENATE/EICOSADIOATE | 1.5 |
| | SYNTHETIC WAX | 6.5 |
| | POLYBUTENE | 1.0 |
| | PROPYL PARABEN | 0.25 |
| | TOCOPHEROL ASCORBYL PALMITATE | 0.05 |
| | LECITHIN GLYCERYL STEARATE | |
| | GLYCERYL OLEATE CITRIC ACID | |
| 2 | POLYETHYLENE | 1 |
| 3 | ISODODECANE | 30.7 |
| | DISTEARDIMONIUM HECTORITE | 4.2 |
| | ALCOHOL | 1.4 |
| 4 | IRON OXIDE | 10 |
| | ISODODECANE | 7 |
| | ETHYLENE/PROPYLENE COPOLYMER | 3 |
| | ISODODECANE | |
| | PANTHENOL WATER | 0.35 |
| 5 | SILICA ETHYLENE/METHACRYLATE | 3 |
| | COPOLYMER ISOPROPYL TITANIUM | |
| | TRIISOSTEARATE | |

This composition can be prepared in the following way:

The ingredients of phase 1 are melted at 95° C., and then phase 2, the gel formed by phase 3, the milled pigments of phase 4 and subsequently phase 5 are added thereto. The product is subsequently hot-cast as a solid eyeliner which has a bouncy texture.

### Example 4

### Foundation

A foundation having the composition indicated in Table 4 below, in which the proportions of the ingredients are expressed as percentages by weight, was manufactured.

TABLE 4

| Phase | INCI NAME/chemical name | % |
|---|---|---|
| 1 | ETHYLENEDIAMINE/HYDROGENATED | 7.20 |
| | DIMER DILINOLEATE COPOLYMER | |
| | BIS-DI-C14-18 ALKYL AMIDE | |
| | GLYCERYL BEHENATE/EICOSADIOATE | 2.00 |
| | SYNTHETIC WAX | 0.50 |
| | ETHYLHEXYL METHOXYCINNAMATE | 7.00 |
| | PHENOXYETHANOL | 0.80 |
| | TOCOPHERYL ACETATE | 0.50 |
| | PEG-8 & TOCOPHEROL & ASCORBYL | 0.20 |
| | PALMITATE & ASCORBIC ACID & CITRIC | |
| | ACID | |
| 2 | OCTYLDODECYL NEOPENTANOATE | 6.60 |
| | Pigments | 7.70 |
| 3 | ETHYLENE/PROPYLENE COPOLYMER & | 12.5 |
| | ISODODECANE | |
| | CAPRYLIC/CAPRIC TRIGLYCERIDE & | 10.0 |
| | STEARALKONIUM HECTORITE & | |
| | PROPYLENE CARBONATE | |
| | CYCLOPENTASILOXANE & C30-45 ALKYL | 5.00 |
| | STEARYL DIMETHICONE CROSSPOLYMER | |
| | ISODODECANE | 12.90 |
| 4 | CYCLOMETHICONE & TITANIUM DIOXIDE | 4.00 |
| | & DIMETHICONE COPOLYOL & ALUMINA & | |
| | METHICONE | |
| 5 | ALUMINUM STARCH OCTENYLSUCCINATE | 6.90 |
| | METHYL METHACRYLATE CROSSPOLYMER | 4.80 |
| | NYLON-12 & HYDROLYZED SOY PROTEIN | 3.00 |
| | & PROPYLENE GLYCOL | |
| | NYLON-12 | 2.80 |
| | MICA & ALUMINUM HYDROXIDE | 3.40 |
| | LAUROYL LYSINE | 2.20 |

This composition can be prepared in the following way: Phase 1 is heated in a water bath at 95° C. with a deflocculator, and then phase 2 is milled in a roller mill. The milled material is added to phase 1. Phase 3 is premixed under cold conditions in a deflocculator, and then added to the mixture of the previous phases. Phase 4 is subsequently added, and the mixture is left to cool to 30° C. in a basin of cold water, still with stirring with a deflocculator, before adding the powders. A very thick and glossy foundation which slides well on application, has good staying power over time and does not migrate into the fine lines around the eyes and the lips, is obtained.

### Example 5

### Sensory Evaluation

The formulation of Example 1, and also the three other similar formulations, were each packaged in a small bottle, and then evaluated by a panel of individuals under standardized temperature, hygrometry and light conditions.

The composition of the four formulations tested is given in Table 5 below.

TABLE 5

| Ingredient | Amount (%) | | | |
|---|---|---|---|---|
| (trade name) | Ex 1 | Ex 2 | Comp. Ex 1 | Comp. Ex 2 |
| SYLVACLEAR A200V | — | 10.56 | 10.56 | 10.56 |
| SYLVACLEAR A2614V | 10.56 | — | — | — |
| CERABEIL⁽¹⁾ | — | — | 10.56 | 10.56 |
| NOMCORT HK-G | 10.56 | 10.56 | 10.56 | — |
| PARAFLINT C80 | 10.56 | 10.56 | — | 10.56 |
| DERMOL DOA | 3.00 | 3.00 | 3.00 | 3.00 |
| SALACOS 222 | 13.70 | 13.70 | 13.70 | 13.70 |
| EUTANOL G | 23.50 | 23.50 | 23.50 | 23.50 |
| POB P | 0.30 | 0.30 | 0.30 | 0.30 |
| PHENOXYETHANOL | 0.10 | 0.10 | 0.10 | 0.10 |
| INDOPOL H-2100 | 7.50 | 7.50 | 7.50 | 7.50 |
| RUBIN LAKE C19003 | 1.26 | 1.26 | 1.26 | 1.26 |
| D&C RED 33 | 1.08 | 1.08 | 1.08 | 1.08 |
| RUBIN LAKE C19025 | 2.00 | 2.00 | 2.00 | 2.00 |
| YELLOW 6 | 1.68 | 1.68 | 1.68 | 1.68 |
| SALACOS 222 | 14.20 | 14.20 | 14.20 | 14.20 |

⁽¹⁾White beeswax

Each of these products was applied to the lower lip of the individual, using the fine applicator brush with which the small bottle is equipped, with the outline of the lip being clearly drawn. The fine brush was then immersed in the composition before applying the product to the upper lip in the same manner.

The panelists evaluated the organoleptic properties of the products on application (gloss and threading when taken from the small bottle), and 1h 30 after application (gloss, staying power and migration), using descriptors with which they were provided and according to a scale of 0 (not) to 5 (very). The gloss was evaluated according to the degree of reflected light, evaluated visually on the lips. The staying power described the ability of the product to adhere to the lips and the migration its capacity to migrate into the perilabial fine lines.

For the migration measurement, the following scale was used:
0: no visible migration even on tightened lips
1: slight visible migration tight lips
2: slight visible migration immobile lips
3: visible migration immobile lips
4: fuzzy appearance of the product around the lips
5: fuzzy appearance of the product around the lips and very visible and considerable migration

The results obtained are given in Table 6 below.

TABLE 6

| | Mean value (/10) | | | |
|---|---|---|---|---|
| Attribute | Ex 1 | Ex 2 | Comp. Ex 1 | Comp. Ex 2 |
| Sheen (T0) | 3 | 4.5 | 4.25 | 4.5 |
| Staying power of | 1.5 | 2 | 2 | 1 |
| the sheen (T1h30) | | | | |
| Staying power | 4.5 | 3 | 2 | 2 |
| Migration (mm) | 0 | 2 | 3 | 3 |
| Threading on | 0 | 0 | 1 | 1.5 |
| removal from the | | | | |
| small bottle | | | | |

It is thus noted that the compositions according to the invention, which have a high pigment content reflected by a covering effect (judged to be 4.5 by the panel) and a strong coloration, migrate much less to the area around the lips, and in particular into the fine lines than the compositions given by way of comparison, which contained only two of the three essential constituents of the compositions according to the invention. The compositions according to the invention also have a better staying power on the lips.

In addition, they do not show any threading when taken from the small bottle that may affect the accuracy of the line drawn on the lips.

These advantages contribute to a sharper line being obtained with the compositions according to the invention.

Finally, it is noted that these advantageous properties are achieved without negatively affecting the other properties desired for lip glosses, namely satisfactory and long-lasting gloss.

### Example 6

### Comparison with a Commercially Available Product

A sensory evaluation similar to that of Example 5 was carried out in order to compare the organoleptic properties of the composition of Example 1 and of a reference product sold by the company Guerlain under the trade name Kiss Kiss Laque®. It is a gloss sold as giving very full coverage and being ultra-glossy.

It appeared that the composition according to the invention provided as high a gloss as and an even greater color strength than the reference composition, without the sharpness of the lip outline being substantially affected.

### Example 7

### Comparison of Example 1 with Comparative Example 3, in which Ester (b) has been Replaced by an Ester Comprising a Diacid (ii) being Outside the Claimed Scope

Composition of example 1 is compared to comparative example 3 in which ester (b) has been replaced by an ester of dilinoleic acid.

Nomcort HK-G used in example 1 has been replaced by Hailucent ISDA that corresponds to polyglyceryl-2 isostearate/dimer dilinoleic acid copolymer; the other ingredients being the same.

Formula of example 1 and formula of comparative example have been tested on a panel in standardized conditions of temperature, hygrometry and light.

The composition of the two formula that have been tested is presented in Table 7 below.

TABLE 7

| | |
|---|---|
| Ingredient | Quantity (%) |

| (nom commercial) | comp. Ex 1 | Ex 3 |
|---|---|---|
| SYLVACLEAR A2614V | 10.56 | 10.56 |
| CERABEIL⁽¹⁾ | — | — |
| NOMCORT HK-G | 10.56 | — |
| HAILUCENT ISDA | | 10.56 |
| PARAFLINT C80 | 10.56 | 10.56 |
| DERMOL DOA | 3.00 | 3.00 |
| SALACOS 222 | 13.70 | 13.70 |
| EUTANOL G | 23.50 | 23.50 |
| POB P | 0.30 | 0.30 |
| PHENOXYETHANOL | 0.10 | 0.10 |
| INDOPOL H-2100 | 7.50 | 7.50 |
| RUBIN LAKE C19003 | 1.26 | 1.26 |
| D&C RED 33 | 1.08 | 1.08 |
| RUBIN LAKE C19025 | 2.00 | 2.00 |
| YELLOW 6 | 1.68 | 1.68 |
| SALACOS 222 | 14.20 | 14.20 |

⁽¹⁾White Beeswax

Each composition was applied on the lower lip of each person, using a paintbrush. The paintbrush is then put again in the composition before making up the upper lip.

Panelists assessed the organoleptic characteristics of the applied compositions in terms of brightness and running out of bottle. 1h30 after application, they have assessed brightness, maintenance and migration, using descriptors that have been provided.

The gloss was visually evaluated according to the degree of reflected light on the lips. Wear describes the ability of the product to adhere on the lips and migration describes the ability of the product to migrate into the peri-labial wrinkles.

The result of this comparative trial is that comparative example 3 runs much easier out of bottle, the result gives a less intense makeup, a weaker wear, and much faster migration than Example 1 of the invention.

1-20. (canceled)

21. A cosmetic composition comprising, in a physiologically acceptable medium:
(a) at least one polyamide resin bearing end groups with a tertiary amide or ester function;
(b) at least one ester obtained from: (i) a saturated or unsaturated, linear or branched, aliphatic acid or hydroxy acid containing from 8 to 30 carbon atoms, (ii) a linear or branched diacid containing from 12 to 36 carbon atoms chosen from eicosadioic acid, ethyloctadecanedioic acid and dodecanedioic acid, and mixtures thereof, and (iii) glycerol or a glycerol condensate; and
(c) at least one apolar wax.

22. The composition as claimed in claim 21, wherein said polyamide resin comprises one or more copolymers corresponding to formula (1): 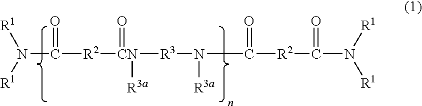
in which:
n denotes a number of repeating units such that the end amide groups represent from 10% to 50% of the total number of amide groups of the resin,
the R¹ groups independently denote C₁-C₂₂ hydrocarbon-based groups,
the R² groups independently denote C₂-C₄₂ hydrocarbon-based groups,
the R³ groups independently denote organic groups containing at least two carbon atoms and optionally one or more oxygen and/or nitrogen atoms, and
the R^{3a} groups independently denote hydrogen, C₁-C₁₀ alkyl groups or a direct bond with R³, or together form a heterocycle including the nitrogen atoms to which they are attached and R³.

23. The composition as claimed in claim 22, wherein said polyamide resin comprises one or more copolymers corresponding to formula (2): 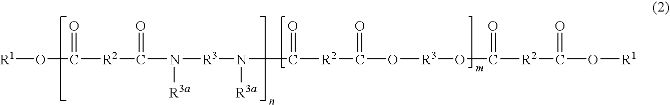
in which:
n denotes a number of repeating units such that the end ester groups represent from 10% to 50% of the total number of amide and ester groups of the resin,
the R¹ groups independently denote C₁-C₂₂ hydrocarbon-based groups,
the R² groups independently denote C₂-C₄₂ hydrocarbon-based groups,
the R³ groups independently denote organic groups containing at least two carbon atoms and optionally one or more oxygen and/or nitrogen atoms, and
the R^{3a} groups independently denote hydrogen, C₁-C₁₀ alkyl groups or a direct bond with R³, or together form a heterocycle including the nitrogen atoms to which they are attached and R³.

24. The composition as claimed in claim 22, wherein n ranges from 1 to 10 and preferably from 1 to 5.

25. The composition as claimed in claim 22, wherein the end amide or ester groups represent from 15% to 40%, preferably from 20% to 35% of the total number of amide and ester groups of the resin.

26. The composition as claimed in claim 22, wherein at least one, and preferably all, of the R¹ groups is (are) alkyl or alkenyl groups containing from 4 to 22 carbon atoms and preferably from 16 to 22 carbon atoms.

27. The composition as claimed in claim 22, wherein at least one, and preferably at least 50% or even all, of the R² groups is (are) alkylene or alkenylene groups containing from 2 to 42, and preferably from 30 to 42 carbon atoms.

28. The composition as claimed in claim 22, wherein R^{3a} denotes hydrogen.

29. The composition as claimed in claim 22, wherein at least one, and preferably all, of the R³ groups contain(s) from 2 to 36 carbon atoms, preferably from 2 to 12, and more preferably from 2 to 8 carbon atoms.

30. The composition as claimed in claim 28, wherein R³ denotes a polyoxyalkylene group such as a polyoxyethylene or polyoxypropylene group.

31. The composition as claimed in claim 28, wherein R³ denotes a polyalkylene amine group such as an —NH—(CH₂CH₂NH)ₚCH₂CH₂—NH— group where p is an integer from 1 to 5.

32. The composition as claimed in claim 21, wherein the resin has a weight-average molecular mass ranging from 500 to 6000, preferably from 4000 to 6000 g/mol, and better still from 4000 to 5000 g/mol, as measured by gel permeation chromatography using calibration with polystyrene.

33. The composition as claimed in claim 21, wherein the aliphatic acid (i) is chosen from lauric acid, myristic acid, palmitic acid, oleic acid, stearic acid, 12-hydroxystearic acid and behenic acid, and mixtures thereof.

34. The composition as claimed in claim 21, wherein the ester (b) is a diester of eicosadioic acid and of glycerol esterified with behenic acid.

35. The composition as claimed in claim 21, wherein the apolar wax is chosen from paraffin wax, polymethylene wax, polyethylene wax, polypropylene wax or ethylene/propylene copolymer wax, microcrystalline waxes and ozokerite, and mixtures thereof.

36. The composition as claimed in claim 21, wherein the polyamide resin represents from 1% to 30% by weight, and preferably from 1% to 20% by weight, more preferably from 5% to 15% by weight, and better still from 8% to 12% by weight, relative to the total weight of the composition.

37. The composition as claimed in claim 21, wherein the ester (b) represents from 0.01% to 30% by weight, preferably from 1% to 20% by weight, more preferably from 5% to 15% by weight, and better still from 8% to 12% by weight, relative to the total weight of the composition.

38. The composition as claimed in claim 21, wherein the apolar wax represents from 0.01% to 40%, preferably from 1% to 30%, more preferably from 5% to 20%, and better still from 8% to 12% by weight, relative to the total weight of the composition.

39. The composition as claimed in claim 21, which is anhydrous.

40. A cosmetic process for making up the lips, comprising the topical application to the lips of the composition as claimed in claim 21.
